(19) United States
(12) Patent Application Publication
(10) Pub. No.: US 2005/0065470 A1
(43) Pub. Date: Mar. 24, 2005

(54) Collection reservoir for an ambulatory pain pump system

(76) Inventors: Pamela S. Reed, Knoxville, TN (US); W. Gilmer Reed, Knoxville, TN (US)

Correspondence Address: Pamela S. Reed, 211 Blount Ave., Suite 509, Knoxville, TN, 37920, US

(21) Appl. No.: 10/930,589

(22) Filed: Aug. 31, 2004

Related U.S. Application Data
(60) Provisional application No. 60/503,748, filed on Sep. 19, 2003.
(60) Provisional application No. 60/507,040, filed on Sep. 29, 2003.

(51) Int. Cl. A61M 1/00

(52) U.S. Cl. 604/122; 604/284

(57) ABSTRACT

An apparatus for the collection of excess delivered local anesthetic from a ambulatory pain pump system into a bulb collection reservoir having a flexible compressible container. A cap is located at one end of the bulb having two ports, an inlet port for accepting excess local anesthetic and an outlet port with a removable closure allowing air to escape as the collection reservoir fills forming a closed system. The apparatus for the collection of excess local anesthetic delivered at a relatively constant controlled rate of flow into an intra-operative site is universal to fit a pain pump system with a fluid storage means, a catheter for deliver of the fluid, and a flow rate restrictor. The collection apparatus may also be attached to the catheter of a pain pump system at the distal end or more proximal by means of a Y or T collector located within the catheter. This safety feature can be universally applied to any pain pump system.

### FIELD OF THE INVENTION

The present invention relates to an apparatus for the collection of excess delivered fluid from a closed ambulatory pain pump system with a relatively constant controlled flow rate of local anesthetic in the form of a bulb reservoir for collecting excess local anesthetic not accepted into a surgical site. The bulb reservoir includes a cap with two ports, an inlet port with semi-permeable cover and opening under the inlet port of the cap for collecting excess local anesthetic not accepted into a surgical site by way of a catheter connected to a pain pump system, and an outlet port with semi-permeable cover under the cap allowing for air to escape from the flexible bulb as fluid enters the collection reservoir. The bulb reservoir cap has a one inlet port with opening through the semi permeable material such as Gortex® to allow fluid to enter the collection reservoir providing a means to receive fluids not accepted into a surgical site from a ambulatory pain pump system and an outlet port covered with a semi permeable material such as Gortex® which is located on the inside of said cap to allow air to escape as fluid enters the collection reservoir.

More particularly this invention relates to a simple, compact, inexpensive collection reservoir which is capable of attaching to a catheter distal to a flow rate restrictor on a ambulatory pain pump system collecting any excess local anesthetic not accepted into a surgical site from a pain pump system at a relatively constant dosage of an active local anesthetic agent at a controlled rate for intra-operative delivery and to a method for achieving such delivery of fluid. The catheter having an inside diameter of 3 mm fitting snugly over the inlet port with an outside diameter of 2.5 mm

The local anesthetic is delivered to the collection reservoir by way of a Y or T connector located distal to the flow rate restrictor on the catheter when the rate of absorption within the surgical site of local anesthetic is less than the rate in which a local anesthetic is being delivered. The local anesthetic is thereby diverted from the surgical site into said collection reservoir by way of a Y or T connector located within the catheter. By way of example, if the ambulatory pain pump is preset to deliver 0.5 cc of local anesthetic per hour and the rate of absorption into a surgical site of local anesthetic is only 0.2 cc of local anesthetic then 0.3 cc of local anesthetic will divert from the surgical site into the collection reservoir by way of a T or Y connector into a collection bulb reservoir.

Intra-operative pressure within the surgical site develops if the rate of infusion of local anesthetic into the surgical site is higher than the rate of absorption. Pressure from the local anesthetic collects within the soft tissue space of a closed surgical site with a dressing applied over the surgical site for compression to control bleeding. The pressure, which builds in the surgical site, force the diversion of local anesthetic by way of a Y or T connector into a collection bulb reservoir preventing any further pressure from building in the surgical site by a constant rate of infusion delivered by the pain pump system. This safety feature can be universally applied to any pain pump system.

An alternative method which may be applied if the flow rate restrictor is controlled by the catheter size is to place the collection reservoir bulb can be placed at the distal end of the catheter. The catheter would exit the surgical site and the collection reservoir attached by means of placing the catheter through the inlet port. It is a further benefit that the collection bulb reservoir may be squeezed with the outlet port uncapped and the cap then placed over the outlet port after evacuation of air within the collection reservoir so as to provide a closed suction system to draw local anesthetic through the surgical site into the collection bulb reservoir. This method provides an additional benefit of suction to the collection reservoir.

The bulb collection reservoir has the capacity of from 150 cc to 450 cc of volume space available sufficient enough to accept the local anesthetic from many current pain pump systems on the market without having to be detached and replaced from the system if the collection reservoir is attached by way of a Y or T connector. The collection bulb may be replaced if needed when attached to the end of a catheter which acts not only to collect excess local anesthetic but body fluids as well. The whole system inclusive of the collection reservoir and pain pump is a single use disposable system.

### BACKGROUND OF THE INVENTION

There are numerous current ambulatory pain pump systems on the market indicated for the intravenous, intra-arterial, subcutaneous and epidural infusion of an active local anesthetic agent with the medication in the form of a fluid preset for a continuous delivery at a controlled infusion rate over a period of time. While the prior art devices describe self-driven ambulatory pain pump systems described in U.S. Pat. Nos. 4,318,400, 4,386,929 and 4,180,067 they do not disclose the feature of the present invention, a collection reservoir which accepts any excess local anesthetic not deliverable into a intra-operative site.

I-Flow, Advanced Infusion, Bard, Abbott, Sorenson and Stryker have commercially marketed ambulatory pain pump systems that deliver a local anesthetic by way of a pump, flow rate restrictor and catheter. The local anesthetic delivered to a surgical site is used for postoperative pain management. Currently, an ambulatory pain pump system is used to manage postoperative pain in orthopedic, general, ob/gyn, podiatry and plastic surgery markets.

These systems have a common pump chamber with storage compartment that holds a local anesthetic which passes through an outlet on the pump connected to a catheter with a flow rate restrictor and a perforated opening(s) at the end of the catheter allowing the passage of a local anesthetic from the catheter into the surgical site at a controlled rate. The rate of infusion is preset by the manufacture to deliver a specific amount of local anesthetic into a surgical site over a period of time.

This has the disadvantage when used in an intra-operative site if the rate of absorption of local infiltrate is less than the rate of infusion resulting in a pooling of fluid within the soft tissue allowing pressure to build up within the soft tissue. The pressure build up within the soft tissue of a surgical site can cause either leakage of local anesthetic through the incision or lead to tissue death by way of tissue necrosis due to the build-up of pressure in the surgical site. The added effect of a compression dressing over the surgical site can further decrease the rate of absorption.

The present invention has the advantage of addressing the potential problem of a pressure build up of local anesthetic within the soft tissue of a surgical site by the addition of a universal collection reservoir added to a closed ambulatory pain pump system to collect local anesthetic that may not be absorbed or accepted within an intra-operative site. An article entitled “Local Anesthetic Infusion Pump Systems Adverse Events Reported to the Food and Drug Administration” in the May, 2003, issue of the American Society of Anesthesiologist, authored by Lori Brown, PhD, an Epidemiologist with the FDA, and Ms. Audrey Morrison, RN, discuss the adverse events in the delivery of to much local anesthetic into a surgical site. The collection reservoir accepts local anesthetic when pressure builds up in the intra-operative from the infusion of local anesthetic. An increase in pressure in the catheter before the local anesthetic will prevent additional local anesthetic from entering the surgical site and divert local anesthetic being pumped into a surgical site to the collection reservoir by way of a Y or T connector or with the collection reservoir located at the distal end of the catheter outside of the surgical site. This described addition of a collection reservoir which is universal would fit any ambulatory pain pump system which is universal and provides a safe means of delivering a local anesthetic into an intra-operative site by a pain pump system.

Thus there remains a need for a simple inexpensive universal collection reservoir which is capable of accepting excess fluid not deliverable at a relatively constant controlled rate of flow which may not be absorbed or accepted into a surgical site at the rate of infusion within a surgical site that may exceed the rate of absorption.

### SUMMARY OF THE INVENTION

The bulb for a collection reservoir in an ambulatory pain pump system according to the present invention provides a flexible compressible bulb having a semi rigid cap on one end thereof with two ports extending through the cap. One is an inlet port with an opening through the semi permeable barrier under the cap permitting the passage of flow of local anesthetic into the bulb but preventing local anesthetic from returning into the catheter passageway. The other port on the semi rigid cap allows the passage of air out of the bulb collection reservoir as the bulb fills with excess local anesthetic not accepted into a surgical site when the rate of absorption is less than the rate of infusion.

The outlet port may be closed by way of a cap attached to the port allowing the bulb collection reservoir to provide suction if squeezed when placed at the distal end of the catheter allowing a local anesthetic and body fluids to be suctioned through the surgical site into the collection chamber. The bulb may be squeezed until it is completely evacuated of air acting and used as a suction device if placed at the end of the catheter with the semi permeable cover preventing the fluid from re-entering the catheter. Placing the cap over the outlet port prevents sucking in air into the bulb and maintains the bulb in a collapsed suction activated state so that only fluid entering the bulb from the closed wound drain will allow the bulb to expand.

Thus, according to the present invention a completely closed system is provided for accepting local anesthetic not accepted into a surgical site under a constant flow rate or local anesthetic and body fluids from a surgical site from a closed wound passing the fluids into a bulb collection reservoir. The collection reservoir bulb should be larger in volume than the amount of local anesthetic to be used and the bulb disposed along with the pain pump system when the desired results of controlling post op pain are achieved.

Accordingly, it is a principal object of the present invention to provide an inexpensive apparatus adaptable to any ambulatory pain pump system for the receiving of excess fluid not accepted into an intraoperative site delivered at a relatively constant controlled rate of flow. It is a related object to provide such an apparatus for receiving a medicinal liquid such as a local anesthetic.

It is a further object of this invention to provide such an apparatus that is useful in receiving excess local anesthetic over a prolonged period of time.

It is yet another object of this invention to provide such an apparatus that is universal to fit with a closed ambulatory pain pump system.

A related object is to provide such a device that is portable, disposable and is thus suitable for use by an ambulatory patient.

It is a further object of the present invention to provide an apparatus for receiving fluid from a relatively constant, controlled rate of flow.

An object of the present invention is to provide a closed wound collection device which includes a bulb collection reservoir having an semi permeable cover within the cap integrally formed therein together with means for allowing the one way passage of local anesthetic an the collecting of fluids within the bulb collection reservoir.

Another object of the present invention is to provide a bulb collection reservoir with one end having an inlet port with one way semi permeable with opening in the cover fashioned by cyclohexanone or other means on the inside of the cap over the inlet port, together with a outlet port located on the cap with a semi permeable cover to allow air to escape as fluid enters the collection bulb reservoir.

Another object of the present invention is to provide a removable cover over the outlet port located on the cap when the bulb is squeezed to provide suction in the collection reservoir allowing body fluids and local anesthetic to enter the bulb collection reservoir in a closed system.

Other objects and many of the intended advantages of the present invention will become more readily apparent upon consideration of the following detailed specification in connection with the accompanying drawings.

### BRIEF DESCRIPTION OF THE DRAWINGS

FIG. 1 is a schematic view of a closed ambulatory pain pump system with the collection reservoir connected by means of a Y or T connector located distal to a flow rate restrictor within the catheter before entering the surgical site.

FIG. 2 is a schematic vies of a closed ambulatory pain pump system with the collection reservoir connected at the distal end of the catheter after exiting the surgical site.

FIG. 3 is an exploded perspective view showing the various elements of the bulb collection reservoir;

FIG. 4 is a plan view of the inlet cap of the bulb evacuator along the line 4-4 of FIG. 1;

FIG. 5 is a sectional view of the cap along the lines 5-5 of FIG. 4;

### DETAILED DESCRIPTION OF THE PREFERRED EMBODIMENT

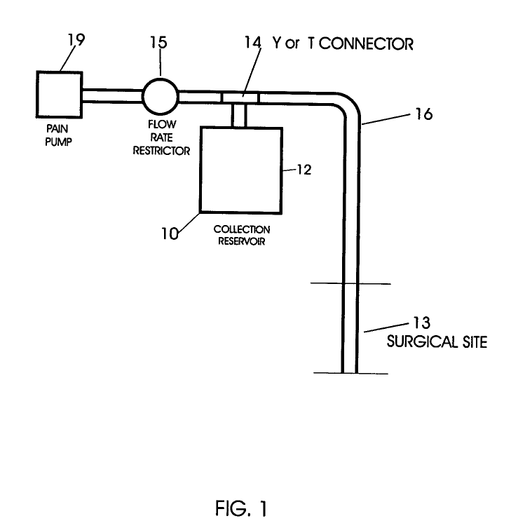

As shown in FIG. 1, the closed wound pain pump 19 system with flow rate restrictor 15 and collection reservoir 10 of the present invention has a closed bulb collection reservoir 12 connected to a catheter 16 by way of a Y or T connector 14 directing the flow of excess local anesthetic not accepted into a surgical site 13 in which the collection reservoir is located proximal to the surgical site. The Y or T connector 14 may be formed of a semi rigid plastic material such as, for example, polyvinyl chloride.

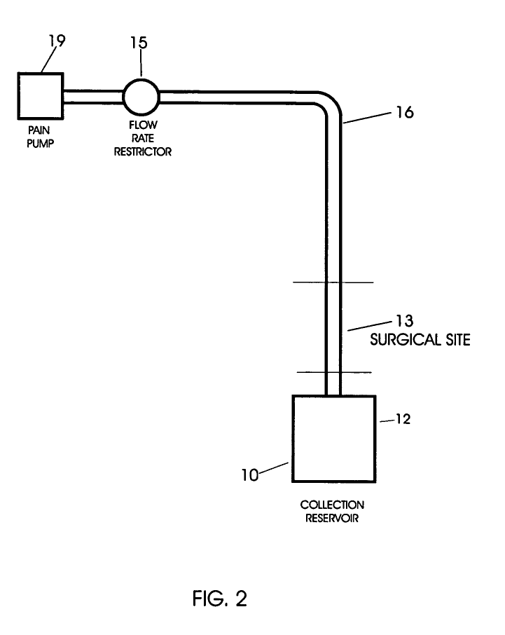

As shown in FIG. 2, the closed wound pain pump 19 system with flow rate restrictor 15 and collection reservoir 10 of the present invention has a closed bulb collection reservoir 12 connected by way of catheter 16 located distal to the surgical site 13. The flow rate restrictor may be used by increasing or decreasing the inside diameter and length of catheter.

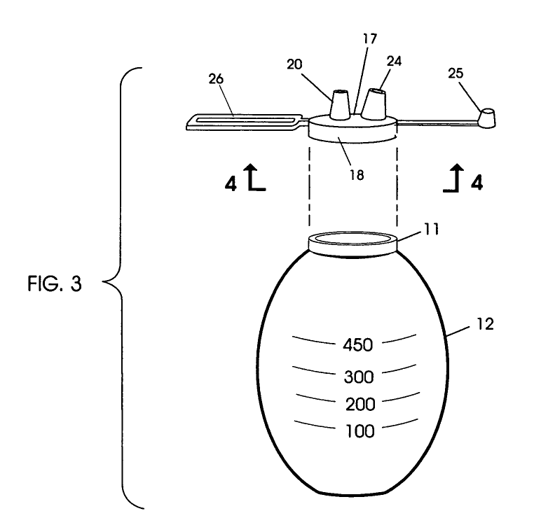

With reference now to the drawings in which like numerals indicate like elements throughout the several views, a presently preferred embodiment of the bulb collection reservoir is depicted in detail in FIG. 3. There is provided a flexible bulb 12 which is generally elliptical in shape and which may be made of a material such as polyvinyl chloride medical grade or silicone rubber. The material used is translucent so that the contents may be viewed. The inlet end of the bulb collection reservoir is formed with a circular opening having an enlarged circular flange 11 surrounding the opening.

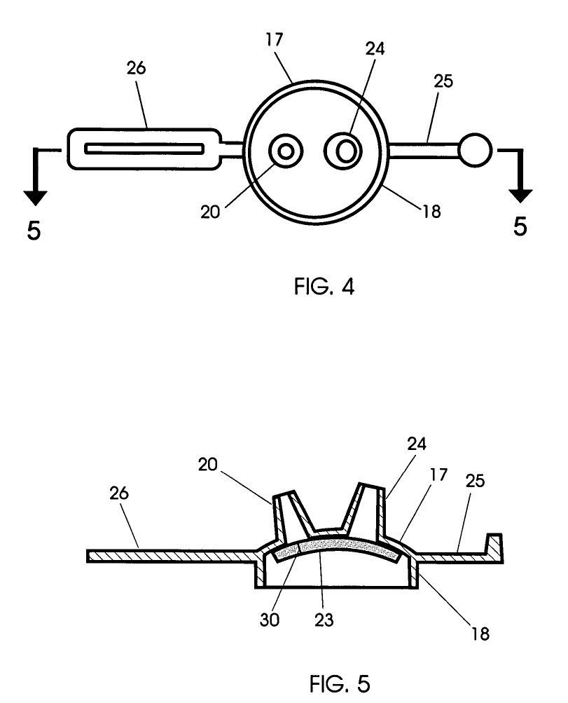

The cap 17 as seen in FIGS. 3, 4 and 5 may be formed of a semi rigid plastic material such as, for example, polyvinyl chloride. The cap 17 has integrally formed spaced circular flange 18 formed thereon as seen in FIGS. 4 and 5. When assembled the flange 18 fits outside the flange 11 of the bulb evacuator 12. The cap member may be bonded to the bulb by any convenient bonding material such as cyclohexanone. The cap 17 has an inlet passageway 20 extending from the outer face thereof as shown in FIG. 3. Inlet passageway 20 is connecter to the catheter 16 to allow passage of excess local anesthetic into the collection bulb reservoir. Each port has a one way cover 23 which is secured to the inside of the cap 17 by a bonding material such as cyclohexanone illustrated in FIG. 5 which allows for the one way flow of fluid into the collection reservoir by way of the inlet portal 20 opening through the semi permeable membrane 30 and air to escape through the outlet portal 24. This semi permeable membrane 23 on the underside of the cap 17 illustrated in FIG. 5 may be made from a material such as Gortex® which permits the one way passage of fluids into the bulb collection reservoir from inlet port 20 through an opening 30 while allowing air to escape through the outlet port 24 as the flow of fluid enters the collection reservoir.

Formed integrally with the cap 17 are the outlet port 24 and inlet port 20 along with a removable closure 25 which when placed over the outlet port 24 after squeezing the collection bulb reservoir 12 that can act as a suction device for local anesthetic and body fluids when placed distal to the surgical site 13 as shown in FIG. 2. When wound closure is complete, the bulb is compressed with suction port 24 open to expel the air, and then sealed with port closure 25. Upon release of the bulb, suction is created within the bulb which serves to drain the closed surgical site and pulls local anesthetic out of the surgical site. There is also provided a strap 26 formed integrally with the cap 17 having a slot therein which may be used to attach the bulb evacuator to the patient.

When emptying and reactivation of the bulb 12 is desired, the bulb is detached from the catheter 16 and replaced with a new sterile collection bulb 12 if necessary. The bulb may remain in place and removed when the entire system is no longer needed for post operative pain management which may be as short as 24 hours and as long as five days. The use of the presently disclosed collection reservoir bulb in conjunction with pain pump systems previously disclosed providing a safety feature allowing for the collection of any excess local anesthetic not accepted into a surgical site in a closed pain pump system.

Obviously many modifications and variations of the present invention are possible in light of the above teachings.

1. A bulb collection reservoir for a closed ambulatory pain pump system located by way of a Y or T connector attached to a catheter. The collection reservoir comprises a flexible bulb, a cap on one end of said bulb, an inlet port in said cap for connecting the interior of said bulb to a closed ambulatory pain pump system, opening through the semi-permeable cover of the inlet port in said cap for permitting fluid flow into said bulb, an outlet passageway in said cap with one way semi-permeable cover allowing air to escape through the outlet port while preventing a local anesthetic from leaving said port by way of a semi-permeable cover to allow local anesthetic to remain in the interior of the collection reservoir bulb.

2. A bulb evacuator according to claim 1 located at the distal end of said catheter without the benefit of a Y or T connector whereby suction is created by squeezing said bulb and capping the air outlet port creating a suction whereby local anesthetic is pulled through an intra-operative site into a collection reservoir along with body fluids which may collect within a surgical site as the flexible bulb expands to accept fluid through the inlet passageway in said cap.
